SEQUENCE LISTING (2 sequences)

SEQ ID NO: 1 - DNA, 1762 bp, Rattus norvegicus
ggcacgaggcactgggctacagttgcctggcttacaggaactggctcttttcctcaagcctcattaagcagcccactccagttcttgatctttgtctcccagtcctgaagtcctttctctccttaggctgcatccacagcccttctaagtggctgtgagcagtttctcagtatgaactgtatatcagacttcttcacctacgagactaccaagtcggtggttgtgaagagctggaccattgggatcatcaaccgagccgtccagctgctgattatctcctactttgtggggtgggttttcttgcatgagaaggcctaccaagtgagggacaccgccattgagtcctcagtagttacaaaggtgaaaggcttcgggcgctatgccaacagagtcatggacgtgtcggattatgtgaccccaccccagggcacctctgtctttgtcatcatcaccaaaatgatcgttactgaaaatcaaatgcaaggattctgtccagagaatgaagagaagtaccgctgtgtgtctgacagccagtgtgggcctgaacgcttcccaggtggggggatcctcaccggccgctgcgtgaactacagctctgttctccggacctgtgagatccagggctggtgccccactgaggtggacaccgtggagatgcctatcatgatggaggctgagaacttcaccattttcatcaagaacagcatccgtttccctctcttcaactttgagaagggaaacctcctgcctaacctcaccgacaaggacataaagaggtgccgcttccaccctgaaaaggccccattttgccccatcttgagggtaggggatgtggttaagtttgctggacaggattttgccaagctggcccgcacgggtggcgttctgggtattaagatcggctgggtgtgcgatctagacaaggcctgggaccagtgcatccctaaatattccttcactcggctggatggagtttctgagaaaagcagtgtttcccctggctacaacttcaggtttgccaaatactataagatggagaacggcagcgagtaccgcacactcctgaaggcttttggcatccgctttgatgtgctggtatatgggaacgctggcaagttcaacatcatccccaccattatcagctcggtggcggccttcacttctgtgggagtgggcactgttctctgtgacatcatcctgctcaatttcctcaaaggggctgatcactacaaagccaggaagtttgaggaggtgactgagacaacactgaagggtactgcgtcaaccaacccagtgttcgccagtgaccaggccactgtggagaagcagtctacagactcaggggcctattctattggtcactagggcctcttcccagggttccatgctcacccttaggctgcagaacctgcaaacaggccactctatctaagcagtcaggggtgggagggggagaagaagggctgctatttctgctgttcaccccaaagactagatccagatatctaggccctcactgttcaacagataggcaatgcttcccactaagacttgaatcttgcctttaccccttgcatgcctcccacctgcttccctggatcccaggacagcagcatccacccctttccaaaggattgagaaaatggtagctaaggttacacccataggacctaccacgtaccaagcacttccacacatattatcccttttcacccttaaaataatcctataaggtagaaaaaaaaaaaaaaaaaaaaaaaaa

SEQ ID NO: 2 - DNA, 1456 bp, Homo sapiens
aattccggagaggggttggaggaatggggagcctgtccctttaagccatgcaatcctagtgagattctctgctcttgatactctggcttcctgtcctgtaggacctccctctcctgaggccaccactgggcccccttctgagtgtcccctgagcactctctcagcatgaactgcatatccgacttcttcacctatgagaccaccaagtcggtggttgtgaagagctggaccatcgggatcatcaaccgagtagttcagcttctgatcatctcctactttgtagggtgggttttcttgcacgagaaggcttaccaggtacgggacacagccattgagtcctcggtggtaaccaaggtgaagggctccggactctacgccaacagagtcatggatgtgtctgattacgtgacgccacctcagggcacctcggtctttgtcatcatcaccaagatgattgttactgaaaatcagatgcaaggattctgcccagagagtgaggagaaataccgctgtgtatcagacagccagtgcgggcctgagcccttgccaggtggggggatcctcactggccgctgcgtgaactacagctctgtgctccggacctgtgagatccagggctggtgccccacggaggtggacacagtggaaacgcccatcatgatggaagctgagaacttcactattttcatcaagaacagcatccgtttccccctcttcaactttgagaagggaaacctccttcccaacctgacagccagggacatgaagacctgccgcttccacccggacaaggaccctttctgccccatcttgcgggtaggggacgtggtcaagtttgcggggcaggattttgccaaactggcgcgcacggggggagttctgggcattaagatcggctgggtgtgcgacttggacaaggcctgggaccagtgcatccccaaatactccttcacccggctcgacagcgtttctgagaaaagcagcgtgtccccaggctacaacttcaggtttgccaagtactacaaaatggaaaatggcagtgagtaccgcaccctcctgaaggcttttggcatccgcttcgacgtgctggtatacgggaatgctggcaagttcaacatcatccccaccatcatcagctctgtggcggcctttacttctgtgggagtgggaactgttctctgtgacatcatcctgctcaacttcctcaagggggccgaccagtacaaagccaagaagtttgaggaggtgaatgagactacgctgaaaatcgcggctttgaccaacccagtgtaccccagcgaccagaccacagcggagaagcagtccaccgattcgggggccttctccataggccactagggcctctttccagggccccacactcacaaaggctccaggcctccccacagaggaccctgcctgagcaagggggcatgggaaatcgaattcctgctgt

(19) United States
(12) Patent Application Publication
(10) Pub. No.: US 2006/0116339 A1
(43) Pub. Date: Jun. 1, 2006

(54) Antisense modulation of purinoreceptor p2x3

(76) Inventors: Samuel J. Shuster, Edina, MN (US); Ulf N.G. Arvidsson, Lidingo (SE); Laura S. Stone, St. Louis Park, MN (US); Hong-Yan Zhang, Sundbyberg (SE); Lucy Vulchanova Hart, St. Paul, MN (US)

Correspondence Address: FISH & RICHARDSON P.C., PO BOX 1022, MINNEAPOLIS, MN, 55440-1022, US

(21) Appl. No.: 10/494,911

(22) PCT Filed: Nov. 8, 2002
(86) PCT No.: PCT/US02/36000

§ 371 Date: Dec. 3, 2004

Related U.S. Application Data
(60) Provisional application No. 60/337,338, filed on Nov. 9, 2001.

(51) Int. Cl. A61K 48/00; C07H 21/02

(52) U.S. Cl. 514/44; 536/23.1

(57) ABSTRACT

The invention relates to antisense oligonucleotides, compositions and methods useful for modulating the expression of P2X³. The compositions comprise antisense oligonucleotides, particularly antisense oligonucleotides targeted to nucleic acids encoding P2X³.

### RELATED APPLICATIONS

This patent application claims priority to U.S. Application No. 60/337,338, filed Nov. 9, 2001.

### TECHNICAL FIELD

This invention relates to antisense oligonucleotides targeted to specific nucleotide sequences. In particular, the invention pertains to antisense oligonucleotides targeted to the nucleic acid encoding the P2X₃ purinoreceptor, and to their use for reducing cellular levels of P2X₃.

### BACKGROUND

The P2X purinoreceptors are a family of ion channels that are activated by extracellular adenosine triphosphate (ATP). Purinoreceptors have been implicated in a variety of biological functions, especially those related to pain sensitivity. The P2X₃ receptor subunit is a member of this family that was originally cloned from rat dorsal root ganglia (Chen et al. (1995) Nature 377:428-431). The nucleotide and amino acid sequences of both rat and human P2X₃ are known (Lewis et al. (1995) Nature 377:432-435; and Garcia-Guzman et al., (1997) Brain Res. Mol. Brain Res. 47:59-66). P2X₃ is involved in afferent pathways controlling urinary bladder volume reflexes. Therefore, inhibiting P2X₃ may have therapeutic potential in the treatment of disorders of urine storage and voiding such as overactive bladder (Cockayne et al., (2000) Nature, 407:1011-5). P2X₃ also is selectively expressed on nociceptive, small diameter sensory neurons (i.e., neurons that are stimulated by pain or injury), consistent with a role in pain sensitivity. A method for reducing the level or activity of P2X₃ therefore would be useful for modulating pain sensation in a subject suffering from chronic pain.

### SUMMARY

Antisense oligonucleotides can be targeted to specific nucleic acid molecules in order to reduce the expression of the target nucleic acid molecules. For example, antisense oligonucleotides directed at the P2X₃ mRNA could be used therapeutically to reduce the level of P2X₃ receptors in a patient suffering from chronic pain. An inherent challenge of generating antisense oligonucleotides, however, is identifying nucleic acid sequences that are useful targets for antisense molecules. Antisense oligonucleotides are often targeted to sequences within a target mRNA based on, for example, the function of the sequences (e.g., the translation start site, coding sequences, etc.). Such approaches often fail because in its native state, mRNA is generally not in a linear conformation. Typically, mRNAs are folded into complex secondary and tertiary structures, rendering sequences on the interior of such folded molecules inaccessible to antisense oligonucleotides. Only antisense molecules directed to accessible portions of an mRNA can effectively contact the mRNA and potentially bring about a desired result. P2X₃ antisense molecules that are useful to reduce levels of P2X₃ and alleviate pain therefore must be directed at accessible mRNA sequences. The invention described herein provides P2X₃ antisense oligonucleotides directed to accessible portions of a P2X₃ mRNA. These antisense oligonucleotides are therapeutically useful for reducing P2X₃ levels.

The invention features isolated antisense oligonucleotides consisting essentially of 10 to 50 nucleotides and compositions containing such antisense oligonucleotides. The oligonucleotide can specifically hybridize within an accessible region of the rat P2X₃ mRNA in its native state, wherein the accessible region is defined by nucleotides 68 through 88, 209 through 230, 235 through 247, 285 through 296, 346 through 355, 383 through 406, 490 through 512, 530 through 543, 553 through 565, 649 through 658, 665 through 679, 727 through 739, 756 through 779, 817 through 856, 874 through 912, 959 through 991, 1028 through 1050, 1087 through 1116, 1145 through 1177, 1237 through 1256, 1266 through 1281, 1297 through 1307, 1314 through 1334, 1339 through 1359, 1434 through 1463, 1523 through 1535, 1630 through 1646, 1677 through 1688, or 1729 through 1741. The antisense oligonucleotide of the invention also can inhibit the production of P2X₃.

The isolated antisense oligonucleotide can specifically hybridize within an accessible region defined by nucleotides 383 through 406, 756 through 779, 490 through 512, or 727 through 739 of SEQ ID NO:1. The isolated antisense oligonucleotide can specifically hybridize within an accessible region defined by nucleotides 384 through 397, 766 through 775, 495 through 510, or 732 through 736 of SEQ ID NO:1. The isolated antisense oligonucleotide can specifically hybridize within an accessible region defined by nucleotides 1434 through 1463, 1237 through 1256, 959 through 991, or 1028 through 1050 of SEQ ID NO:1. The isolated antisense oligonucleotide can specifically hybridize within an accessible region defined by nucleotides 817 through 856, 553 through 565, 285 through 296, 209 through 230, or 1145 through 1177 of SEQ ID NO:1. The isolated antisense oligonucleotide can specifically hybridize within an accessible region defined by nucleotides 383 through 404, 721 through 744, 747 through 770, or 1314 through 1344 of SEQ ID NO:1.

In some embodiments, compositions include a plurality of isolated antisense oligonucleotides, wherein each antisense oligonucleotide specifically hybridizes within a different accessible region.

The invention also features an isolated antisense oligonucleotide consisting essentially of 10 to 50 nucleotides, wherein the oligonucleotide specifically hybridizes within an accessible region, wherein the region is defined by nucleotides 7 through 29, 95 through 105, 207 through 217, 221 through 240, 248 through 258, 278 through 293, 338 through 365, 471 through 482, 486 through 502, 544 through 562, 747 through 761, 784 through 796, 815 through 850, 865 through 879, 883 through 905, 922 through 932, 953 through 968, 985 through 1000, 1033 through 1044, 1156 through 1170, 1239 through 1261, 1297 through 1314, or 1411 through 1439 of SEQ ID NO:2, and wherein the isolated antisense oligonucleotide inhibits the production of P2X₃. The isolated antisense oligonucleotide can specifically hybridize within an accessible region defined by nucleotides 953 through 968, 1297 through 1314, or 815 through 850 of SEQ ID NO:2; an accessible region defined by nucleotides 957 through 967, 1297 through 1301, or 817 through 823 of SEQ ID NO:2; an accessible region defined by nucleotides 747 through 761, 985 through 1000, or 486 through 502 of SEQ ID NO:2; an accessible region defined by nucleotides 338 through 365, 278 through 293, 544 through 562, or 221 through 240 of SEQ ID NO:2; or within an accessible region defined by nucleotides 484 through 501 or 742 through 762 of SEQ ID NO:2.

The invention also features compositions containing such isolated antisense oligonucleotides. The composition can include a plurality of isolated antisense oligonucleotides, wherein each antisense oligonucleotide specifically hybridizes with a different accessible region.

In another aspect, the invention features an isolated oligonucleotide consisting essentially of the sequence of SEQ ID NO:3, SEQ ID NO:4, SEQ ID NO:5, SEQ ID NO:6, SEQ ID NO:7, or SEQ ID NO:8.

In yet another aspect, the invention features a method of decreasing production of P2X₃ in cells or tissues. The method includes contacting the cells or tissues with an antisense oligonucleotide that specifically hybridizes within an accessible region of P2X₃ provided that the accessible region is not a region defined by nucleotides 1279 through 1296 or 1315 through 1334 of SEQ ID NO:2. The contacting step can result in an inhibition of pain sensory neurons, or can result in increased bladder capacity.

The invention also features an nucleic acid construct that includes a regulatory element operably linked to a nucleic acid encoding a transcript, wherein the transcript specifically hybridizes within one or more accessible regions of P2X₃ mRNA in its native form, and host cells containing such nucleic acid constructs.

In yet another aspect, the invention features an isolated antisense oligonucleotide that specifically hybridizes within an accessible region of P2X₃ mRNA in its native form, provided that the accessible region is not a region defined by nucleotides 1279 through 1296 or 1315 through 1334 of SEQ ID NO:2, and wherein the antisense oligonucleotide inhibits production of P2X₃.

Unless otherwise defined, all technical and scientific terms used herein have the same meaning as commonly understood by one of ordinary skill in the art to which this invention belongs. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is the nucleotide sequence of rat P2X₃ (SEQ ID NO: 1).

FIG. 2 is the nucleotide sequence of human P2X₃ (SEQ ID NO:2).

FIG. 3A and FIG. 3B are line graphs showing the result of nociceptive testing in rats, after catheterization but before induction of chronic neuropathic pain, after induction of chronic neuropathic pain but before antisense treatment, and after antisense treatment. FIG. 3A depicts the results in rats subjected to a mechanical stimulus, and FIG. 3B depicts the results in rats subjected to a thermal stimulus.

FIG. 4A and FIG. 4B are line graphs showing the result of nociceptive testing in rats, after catheterization but before induction of chronic neuropathic pain, after induction of chronic neuropathic pain but before antisense treatment, and after antisense treatment. FIG. 4A depicts the results in rats subjected to a mechanical stimulus, and FIG. 4B depicts the results in rats subjected to a thermal stimulus.

FIG. 5A and FIG. 5B are line graphs showing the results of nociceptive testing in rats, after catheterization but before induction of chronic inflammatory pain, after induction of chronic inflammatory pain but before antisense treatment, and after antisense treatment. FIG. 5A depicts the results in rats subjected to a mechanical stimulus, and FIG. 5B depicts the results in rats subjected to a thermal stimulus.

FIG. 6A, FIG. 6A′, FIG. 6B, and FIG. 6B′ are photographs showing the immunolocalization of P2X₃ in human spinal cord (FIG. 6A and FIG. 6A′) and human dorsal root ganglia (FIG. 6B and FIG. 6B′). For control experiments (shown in FIG. 6A′ and FIG. 6B′), P2X₃ antibodies were pre-incubated with the peptide antigen.

### DETAILED DESCRIPTION

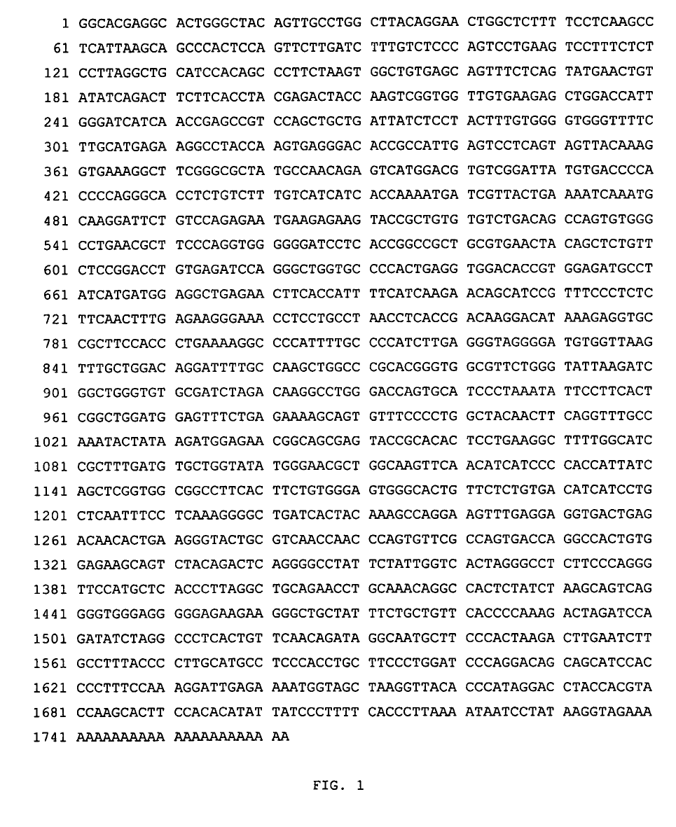

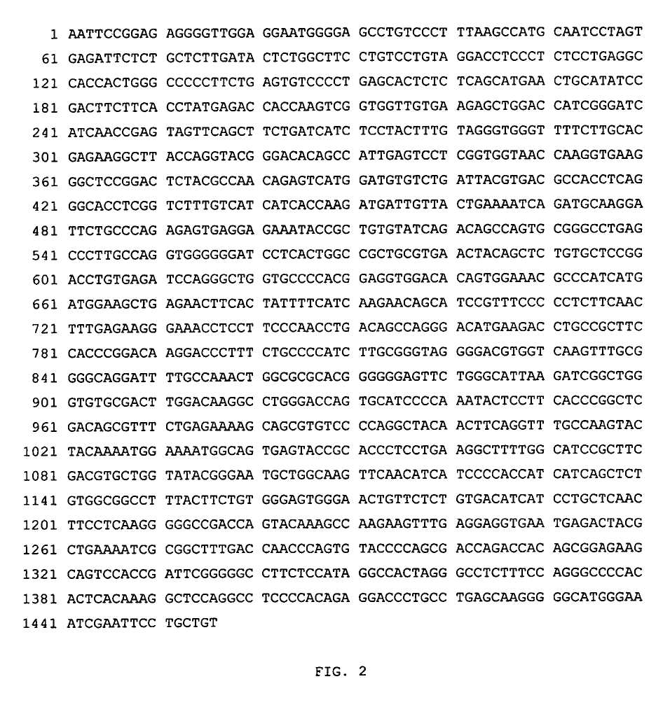

The present invention employs antisense compounds, particularly oligonucleotides, to modulate the function of target nucleic acid molecules. As used herein, the term “target nucleic acid” refers to both RNA and DNA, including cDNA, genomic DNA, and synthetic (e.g., chemically synthesized) DNA. The target nucleic acid can be double-stranded or single-stranded (i.e., a sense or an antisense single strand). In some embodiments, the target nucleic acid encodes a P2X₃ polypeptide. Thus, a “target nucleic acid” encompasses DNA encoding P2X₃, RNA (including pre-mRNA and mRNA) transcribed from such DNA, and also cDNA derived from such RNA. FIGS. 1 and 2 provide nucleic acid sequences that encode rat and human P2X₃ polypeptides, respectively (SEQ ID NO:1 and SEQ ID NO:2, respectively). An “antisense” compound is a compound containing nucleic acids or nucleic acid analogs that can specifically hybridize to a target nucleic acid, and the modulation of expression of a target nucleic acid by an antisense oligonucleotide is generally referred to as “antisense technology”.

The term “hybridization,” as used herein, means hydrogen bonding, which can be Watson-Crick, Hoogsteen, or reversed Hoogsteen hydrogen bonding, between complementary nucleoside or nucleotide bases. For example, adenine and thymine, and guanine and cytosine, respectively, are complementary nucleobases (often referred to in the art simply as “bases”) that pair through the formation of hydrogen bonds. “Complementary,” as used herein, refers to the capacity for precise pairing between two nucleotides. For example, if a nucleotide at a certain position of an oligonucleotide is capable of hydrogen bonding with a nucleotide in a target nucleic acid molecule, then the oligonucleotide and the target nucleic acid are considered to be complementary to each other at that position. The oligonucleotide and the target nucleic acid are complementary to each other when a sufficient number of corresponding positions in each molecule are occupied by nucleotides that can hydrogen bond with each other. Thus, “specifically hybridizable” is used to indicate a sufficient degree of complementarity or precise pairing such that stable and specific binding occurs between the oligonucleotide and the target nucleic acid.

It is understood in the art that the sequence of an antisense oligonucleotide need not be 100% complementary to that of its target nucleic acid to be specifically hybridizable. An antisense oligonucleotide is specifically hybridizable when (a) binding of the oligonucleotide to the target nucleic acid interferes with the normal function of the target nucleic acid, and (b) there is sufficient complementarity to avoid non-specific binding of the antisense oligonucleotide to non-target sequences under conditions in which specific binding is desired, i.e., under conditions in which in vitro assays are performed or under physiological conditions for in vivo assays or therapeutic uses.

Stringency conditions in vitro are dependent on temperature, time, and salt concentration (see, e.g., Sambrook et al., Molecular Cloning: A Laboratory Manual, Cold Spring Harbor Laboratory Press, NY (1989)). Typically, conditions of high to moderate stringency are used for specific hybridization in vitro, such that hybridization occurs between substantially similar nucleic acids, but not between dissimilar nucleic acids. Specific hybridization conditions are hybridization in 5×SSC (0.75 M sodium chloride/0.075 M sodium citrate) for 1 hour at 40° C. with shaking, followed by washing 10 times in 1×SSC at 40° C. and 5 times in 1×SSC at room temperature. Oligonucleotides that specifically hybridize to a target nucleic acid can be identified by recovering the oligonucleotides from the oligonucleotide/target hybridization duplexes (e.g., by boiling) and sequencing the recovered oligonucleotides.

In vivo hybridization conditions consist of intracellular conditions (e.g., physiological pH and intracellular ionic conditions) that govern the hybridization of antisense oligonucleotides with target sequences. In vivo conditions can be mimicked in vitro by relatively low stringency conditions, such as those used in the RiboTAG™ technology described below. For example, hybridization can be carried out in vitro in 2×SSC (0.3 M sodium chloride/0.03 M sodium citrate), 0.1% SDS at 37° C. A wash solution containing 4×SSC, 0.1% SDS can be used at 37° C., with a final wash in 1×SSC at 45° C.

The specific hybridization of an antisense molecule with its target nucleic acid can interfere with the normal function of the target nucleic acid. For a target DNA nucleic acid, antisense technology can disrupt replication and transcription. For a target RNA nucleic acid, antisense technology can disrupt, for example, translocation of the RNA to the site of protein translation, translation of protein from the RNA, splicing of the RNA to yield one or more mRNA species, and catalytic activity of the RNA. The overall effect of such interference with target nucleic acid function is, in the case of a nucleic acid encoding P2X₃, modulation of the expression of P2X₃. In the context of the present invention, “modulation” means a decrease in the expression of a gene (e.g., due to inhibition of transcription) and/or a decrease in cellular levels of the protein (e.g., due to inhibition of translation).

Identification of Target Sequences for P2X₃ Antisense Oligonucleotides

Antisense oligonucleotides are preferably directed at specific targets within a nucleic acid molecule. The process of “targeting” an antisense oligonucleotide to a particular nucleic acid usually begins with the identification of a nucleic acid sequence whose function is to be modulated. This nucleic acid sequence can be, for example, a gene (or mRNA transcribed from the gene) whose expression is associated with a particular disorder or disease state.

The targeting process also includes the identification of a site or sites within the target nucleic acid molecule where an antisense interaction can occur such that the desired effect, e.g., detection of P2X₃ mRNA or modulation of P2X₃ expression, will result. Traditionally, preferred target sites for antisense oligonucleotides have included the regions encompassing the translation initiation or termination codon of the open reading frame (ORF) of the gene. In addition, the ORF has been targeted effectively in antisense technology, as have the 5′ and 3′ untranslated regions. Furthermore, antisense oligonucleotides have been successfully directed at intron regions and intron-exon junction regions.

Simple knowledge of the sequence and domain structure (e.g., the location of translation initiation codons, exons, or introns) of a target nucleic acid, however, is generally not sufficient to ensure that an antisense oligonucleotide directed to a specific region will effectively bind to and modulate the function of the target nucleic acid. In its native state, an mRNA molecule is folded into complex secondary and tertiary structures, and sequences that are on the interior of such structures are inaccessible to antisense oligonucleotides. For maximal effectiveness, antisense oligonucleotides can be directed to regions of a target mRNA that are most accessible, i.e., regions at or near the surface of a folded mRNA molecule.

Accessible regions of an mRNA molecule can be identified by methods known in the art, including the use of RiboTAG™ technology. This technology is disclosed in PCT application number SE01/02054. In the RiboTAG™ method, also known as mRNA Accessible Site Tagging (MAST), oligonucleotides that can interact with a test mRNA in its native state (i.e., under physiological conditions) are selected and sequenced, thus leading to the identification of regions within the test mRNA that are accessible to antisense molecules. In a version of the RiboTAG™ protocol, the test mRNA is produced by in vitro transcription and is then immobilized, for example by covalent or non-covalent attachment to a bead or a surface (e.g., a magnetic bead). The immobilized test mRNA is then contacted by a population of oligonucleotides, wherein a portion of each oligonucleotide contains a different, random sequence. Oligonucleotides that can hybridize to the test mRNA under conditions of low stringency are separated from the remainder of the population (e.g., by precipitation of the magnetic beads). The selected oligonucleotides then can be amplified and sequenced; these steps of the protocol are facilitated if the random sequences within each oligonucleotide are flanked on one or both sides by known sequences that can serve as primer binding sites for PCR amplification.

In general, oligonucleotides that are useful in RiboTAG™ technology contain between 15 and 18 random bases, flanked on either side by known sequences. These oligonucleotides are contacted by the test mRNA under conditions that do not disrupt the native structure of the mRNA (e:g., in the presence of medium pH buffering, salts that modulate annealing, and detergents and/or carrier molecules that minimize non-specific interactions). Typically, hybridization is carried out at 37 to 40° C., in a solution containing 1× to 5×SSC and about 0.1% SDS. Non-specific interactions can be minimized further by blocking the known sequence(s) in each oligonucleotide with the primers that will be used for PCR amplification of the selected oligonucleotides.

As described herein, accessible regions of the nucleic acids encoding human and rat P2X₃ have been mapped. Thus, antisense oligonucleotides of the invention can specifically hybridize within one or more accessible regions defined by: nucleotides 68 through 88, 209 through 230, 235 through 247, 285 through 296, 346 through 355, 383 through 406, 490 through 512, 530 through 543, 553 through 565, 649 through 658, 665 through 679, 727 through 739, 756 through 779, 817 through 856, 874 through 912, 959 through 991, 1028 through 1050, 1087 through 1116, 1145 through 1177, 1237 through 1256, 1266 through 1281, 1297 through 1307, 1314 through 1334, 1339 through 1359, 1434 through 1463, 1523 through 1535, 1630 through 1646, 1677 through 1688, or 1729 through 1741 of SEQ ID NO:1. Particularly useful antisense oligonucleotides include those that specifically hybridize within accessible regions defined by nucleotides 383 through 406 (e.g., 384 through 397), 756 through 779 (e.g., 766 through 775), 490 through 512 (e.g., 495 through 510), 727 through 739 (e.g., 732 through 736), 1434 through 1463, 1237 through 1256, 959 through 991, 1028 through 1050, 817 through 856, 553 through 565, 285 through 296, 209 through 230, or 1145 through 1177 of SEQ ID NO:1.

Antisense oligonucleotides also can specifically hybridize within accessible regions defined by: nucleotides 7 through 29, 95 through 105, 207 through 217, 221 through 240, 248 through 258, 278 through 293, 338 through 365, 471 through 482, 486 through 502, 544 through 562, 747 through 761, 784 through 796, 815 through 850, 865 through 879, 883 through 905, 922 through 932, 953 through 968, 985 through 1000, 1033 through 1044, 1156 through 1170, 1239 through 1261, 1297 through 1314, or 1411 through 1439 of SEQ ID NO:2. Particularly useful antisense oligonucleotides include those that specifically hybridize within accessible regions defined by nucleotides 953 through 968 (e.g., 957 through 967), 1297 through 1315 (e.g., 1297 through 1301), 815 through 850 (e.g., 817 through 823), 747 through 761, 985 through 1000, 486 through 502, 338 through 365, 278 through 293, 544 through 562, or 221 through 240 of SEQ ID NO:2.

Non-limiting examples of such antisense oligonucleotides include the following nucleotide sequences: 5′-GAC ACG TCC ATG ACT CTG TTG G-3′ (SEQ ID NO:3); 5′-GAG GTT TCC CTT CTC AAA-3′ (SEQ ID NO:4); 5′-TGT CCT TGT CGG TGA GGT TAG-3′ (SEQ ID NO:5); 5′-GTA GAC TGC TTC TCC ACA GTG-3′ (SEQ ID NO:6); 5′-CTC CTC ACT CTC TGG GCA-3′ (SEQ ID NO:7); and 5′-GTC CCT GGC TGT CAG GTT GGG A-3′ (SEQ ID NO:8).

It should be noted that an antisense oligonucleotide may consist essentially of a nucleotide sequence that specifically hybridizes with an accessible region set out above. Such antisense oligonucleotides, however, may contain additional flanking sequences of 5 to 10 nucleotides at either end. Flanking sequences can include, for example, additional sequence of the target nucleic acid or primer sequence.

For maximal effectiveness, further criteria can be applied to the design of antisense oligonucleotides. Such criteria are well known in the art, and are widely used, for example, in the design of oligonucleotide primers. These criteria include the lack of predicted secondary structure of a potential antisense oligonucleotide, an appropriate G and C nucleotide content (e.g., approximately 50%), and the absence of sequence motifs such as single nucleotide repeats (e.g., GGGG runs).

P2X₃ Antisense Oligonucleotides

Once one or more target sites have been identified, antisense oligonucleotides can be synthesized that are sufficiently complementary to the target (i.e., that hybridize with sufficient strength and specificity to give the desired effect). In the context of the present invention, the desired effect is the modulation of P2X₃ expression such that cellular P2X₃ levels are reduced. The effectiveness of an antisense oligonucleotide to modulate expression of a target nucleic acid can be evaluated by measuring levels of the mRNA or protein products of the target nucleic acid (e.g., by Northern blotting, RT-PCR, Western blotting, ELISA, or immunohistochemical staining).

In some embodiments, it may be useful to target multiple accessible regions of a target nucleic acid. In such embodiments, multiple antisense oligonucleotides can be used that each specifically hybridize to a different accessible region. Multiple antisense oligonucleotides can be used together or sequentially.

The antisense oligonucleotides in accordance with this invention can be from about 10 to about 50 nucleotidesin length (e.g., 12 to 40, 14 to 30, or 15 to 25 nucleotides in length). Antisense oligonucleotides that are 15 to 23 nucleotides in length are particularly useful. However, an antisense oligonucleotide containing even fewer than 10 nucleotides (for example, a portion of one of the preferred antisense oligonucleotides) is understood to be included within the present invention so long as it demonstrates the desired activity of inhibiting expression of the P2X ₃ purinoreceptor.

An “antisense oligonucleotide” can be an oligonucleotide as described herein. The term “oligonucleotide” refers to an oligomer or polymer of ribonucleic acid (RNA) or deoxyribonucleic acid (DNA) or analogs thereof. This term includes oligonucleotides composed of naturally occurring nucleobases, sugars and covalent internucleoside (backbone) linkages, as well as oligonucleotides having non-naturally occurring portions which function similarly. Such modified or substituted oligonucleotides are often preferred over native forms because of desirable properties such as, for example, enhanced cellular uptake, enhanced affinity for a nucleic acid target, and increased stability in the presence of nucleases.

While antisense oligonucleotides are a preferred form of antisense compounds, the present invention includes other oligomeric antisense compounds, including but not limited to, oligonucleotide analogs such as those described below. As is known in the art, a nucleoside is a base-sugar combination, wherein the base portion is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. The respective ends of this linear polymeric structure can be further joined to form a circular structure, although linear structures are generally preferred. Within the oligonucleotide structure, the phosphate groups are commonly referred to as forming the internucleoside backbone of the oligonucleotide. The normal linkage or backbone of RNA and DNA is a 3′ to 5′ phosphodiester linkage.

P2X₃ antisense oligonucleotides that are useful in the present invention include oligonucleotides containing modified backbones or non-natural internucleoside linkages. As defined herein, oligonucleotides having modified backbones include those that have a phosphorus atom in the backbone and those that do not have a phosphorus atom in the backbone. For the purposes of this specification, and as sometimes referenced in the art, modified oligonucleotides that do not have a phosphorus atom in their internucleoside backbone also can be considered to be oligonucleotides.

Modified oligonucleotide backbones can include, for example, phosphorothioates, chiral phosphorothioates, phosphorodithioates, phosphotriesters, aminoalkylphosphotri-esters, methyl and other alkyl phosphonates (e.g., 3′-alkylene phosphonates and chiral phosphonates), phosphinates, phosphoramidates (e.g., 3′-amino phosphoramidate and aminoalkylphosphoramidates), thionophosphoramidates, thionoalkylphosphonates, thionoalkylphosphotriesters, and boranophosphates having normal 3′-5′ linkages, as well as 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. References that teach the preparation of such modified backbone oligonucleotides are provided, for example, in U.S. Pat. Nos. 4,469,863 and 5,750,666.

P2X₃ antisense molecules with modified oligonucleotide backbones that do not include a phosphorus atom therein can have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. References that teach the preparation of such modified backbone oligonucleotides are provided, for example, in U.S. Pat. Nos. 5,235,033 and 5,596,086.

In another embodiment, a P2X₃ antisense compound can be an oligonucleotide analog, in which both the sugar and the internucleoside linkage (i.e., the backbone) of the nucleotide units are replaced with novel groups, while the base units are maintained for hybridization with an appropriate nucleic acid target. One such oligonucleotide analog that has been shown to have excellent hybridization properties is referred to as a peptide nucleic acid (PNA). In PNA compounds, the sugar-backbone of an oligonucleotide is replaced with an amide containing backbone (e.g., an aminoethylglycine backbone). The nucleobases are retained and are bound directly or indirectly to aza nitrogen atoms of the amide portion of the backbone. References that teach the preparation of such modified backbone oligonucleotides are provided, for example, in Nielsen et al., Science 254:1497-1500 (1991), and in U.S. Pat. No. 5,539,082.

Other useful P2X₃ antisense oligonucleotides can have phosphorothioate backbones and oligonucleosides with heteroatom backbones, and in particular CH₂NHOCH₂, CH₂N(CH₃)OCH₂, CH₂ON(CH₃)CH₂, CH₂N(CH₃)N(CH₃)CH₂, and ON(CH₃)CH₂CH₂ (wherein the native phosphodiester backbone is represented as OPOCH₂) as taught in U.S. Pat. No.5,489,677, and the amide backbones disclosed in U.S. Pat. No. 5,602,240.

Substituted sugar moieties also can be included in modified oligonucleotides. P2X₃ antisense oligonucleotides of the invention can comprise one or more of the following at the 2′ position: OH; F; O—, S—, or N-alkyl; O—, S—, or N-alkenyl; O—, S—, or N-alkynyl; or O-alkyl-O-alkyl, wherein the alkyl, alkenyl and alkynyl can be substituted or unsubstituted C₁ to C₁₀ alkyl or C₂ to C₁₀ alkenyl and alkynyl. Useful modifications also can include O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(C₂)ₙCH₃)]₂, where n and m are from 1 to about 10. In addition, oligonucleotides can comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, groups for improving the pharmacokinetic or pharmacodynamic properties of an oligonucleotide, and other substituents having similar properties. Other useful modifications include an alkoxyalkoxy group, e.g., 2′-methoxyethoxy (2′-OCH₂CH₂OCH₃), a dimethylaminooxyethoxy group (2′-O(CH₂)₂ON(CH₃)₂), or a dimethylamino-ethoxyethoxy group (2′-OCH₂OCH₂N(CH₂)₂). Other modifications can include 2′-methoxy (2′-OCH₃), 2′-aminopropoxy (2′-OCH₂CH₂CH₂NH₂), or 2′-fluoro (2′-F). Similar modifications also can be made at other positions within the oligonucleotide, such as the 3′ position of the sugar on the 3′ terminal nucleotide or in 2′-5′ linked oligonucleotides, and the 5′ position of the 5′ terminal nucleotide. Oligonucleotides also can have sugar mimetics such as cyclobutyl moieties in place of the pentofuranosyl group. References that teach the preparation of such substituted sugar moieties include U.S. Pat. Nos. 4,981,957 and 5,359,044.

Useful P2X₃ antisense oligonucleotides also can include nucleobase modifications or substitutions. As used herein, “unmodified” or “natural” nucleobases include the purine bases adenine (A) and guanine (G), and the pyrimidine bases thymine (T), cytosine (C), and uracil (U). Modified nucleobases can include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil(pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Other useful nucleobases include those disclosed, for example, in U.S. Pat. No. 3,687,808.

Certain nucleobase substitutions can be particularly useful for increasing the binding affinity of the antisense oligonucleotides of the invention. For example, 5-methylcytosine substitutions have been shown to increase nucleic acid duplex stability by 0.6 to 1.2° C. (Sanghvi, Y. S., Crooke, S. T. and Lebleu, B., eds., Antisense Research and Applications, pp. 276-278, CRC Press, Boca Raton, Fla. (1993)). Other useful nucleobase substitutions include 5-substituted pyrimidines, 6-azapyrimidines and N-2, N-6 and O-6 substituted purines such as 2-aminopropyladenine, 5-propynyluracil and 5-propynylcytosine.

Antisense oligonucleotides of the invention also can be modified by chemical linkage to one or more moieties or conjugates that enhance the activity, cellular distribution or cellular uptake of the oligonucleotide. Such moieties include but are not limited to lipid moieties (e.g., a cholesterol moiety); cholic acid; a thioether moiety (e.g., hexyl-S-tritylthiol); a thiocholesterol moiety; an aliphatic chain (e.g., dodecandiol or undecyl residues); a phospholipid moiety (e.g., di-hexadecyl-rac-glycerol or triethyl-ammonium 1,2-di-O-hexadecyl-rac-glycero-3-H-phosphonate); a polyamine or a polyethylene glycol chain; adamantane acetic acid; a palmityl moiety; or an octadecyl amine or hexylamino-carbonyl-oxycholesterol moiety. The preparation of such oligonucleotide conjugates is disclosed in, for example, U.S. Pat. Nos. 5,218,105 and 5,214,136.

It is not necessary for all nucleobase positions in a given antisense oligonucleotide to be uniformly modified. More than one of the aforementioned modifications can be incorporated into a single oligonucleotide or even at a single nucleoside within an oligonucleotide. The present invention also includes antisense oligonucleotides that are chimeric oligonucleotides. “Chimeric” antisense oligonucleotides can contain two or more chemically distinct regions, each made up of at least one monomer unit (e.g., a nucleotide in the case of an oligonucleotide). Chimeric oligonucleotides typically contain at least one region wherein the oligonucleotide is modified so as to confer, for example, increased resistance to nuclease degradation, increased cellular uptake, and/or increased affinity for the target nucleic acid. For example, a region of a chimeric oligonucleotide can serve as a substrate for an enzyme such as RNase H, which is capable of cleaving the RNA strand of an RNA:DNA duplex such as that formed between a target mRNA and an antisense oligonucleotide. Cleavage of such a duplex by RNase H, therefore, can greatly enhance the effectiveness of an antisense oligonucleotide.

The P2X₃ antisense oligonucleotides of the invention are synthesized in vitro and do not include antisense compositions of biological origin, except for oligonucleotides that comprise the subject antisense oligonucleotides and that have been purified from or isolated from biological material. Antisense oligonucleotides used in accordance with this invention can be conveniently produced through the well-known technique of solid phase synthesis. Equipment for such synthesis is commercially available from several vendors including, for example, Applied Biosystems (Foster City, Calif.). Any other means for such synthesis known in the art additionally or alternatively can be employed. Similar techniques also can be used to prepare modified oligonucleotides such as phosphorothioates or alkylated derivatives.

Antisense Preparations and Methods for Use

The antisense oligonucleotides of the invention are useful for research and diagnostics, and for therapeutic use. For example, assays based on hybridization of antisense oligonucleotides to nucleic acids encoding P2X₃ can be used to evaluate levels of P2X₃ in a tissue sample. Hybridization of the antisense oligonucleotides of the invention with a nucleic acid encoding P2X₃ can be detected by means known in the art. Such means can include conjugation of an enzyme to the antisense oligonucleotide, radiolabeling of the antisense oligonucleotide, or any other suitable means of detection.

Those of skill in the art can harness the specificity and sensitivity of antisense technology for therapeutic use. Antisense oligonucleotides have been employed as therapeutic moieties in the treatment of disease states in animals, including humans. For therapeutic methods, the cells or tissues are typically within a vertebrate (e.g., a mammal such as a human.

The invention provides therapeutic methods for treating conditions arising from abnormal expression (e.g., over-production) of the P2X₃ purinoreceptor. By these methods, antisense oligonucleotides in accordance with the invention are administered to a subject (e.g., a human) suspected of having a disease or disorder (e.g., chronic pain, overactive bladder, or irritable bowel syndrome) that can be alleviated by modulating the expression of P2X₃. Typically, one or more antisense oligonucleotides can be administered to a subject suspected of having a disease or condition associated with the expression of P2X₃. The antisense oligonucleotide can be in a pharmaceutically acceptable carrier or diluent, and can be administered in amounts and for periods of time that will vary depending upon the nature of the particular disease, its severity, and the subject's overall condition. Typically, the antisense oligonucleotide is administered in an inhibitory amount (i.e., in an amount that is effective for inhibiting the production of P2X₃ in the cells or tissues contacted by the antisense oligonucleotides). The antisense oligonucleotides and methods of the invention also can be used prophylactically, e.g., to minimize pain in a subject known to have high levels of P2X₃.

The ability of a P2X₃ antisense oligonucleotide to inhibit expression and/or production of P2X₃ can be assessed, for example, by measuring levels of P2X₃ mRNA or protein in a subject before and after treatment. Methods for measuring mRNA and protein levels in tissues or biological samples are well known in the art. If the subject is a research animal, for example, P2X₃ levels in the brain can be assessed by in situ hybridization or immunostaining following euthanasia. Indirect methods can be used to evaluate the effectiveness of P2X₃ antisense oligonucleotides in live subjects. For example, reduced expression of P2X₃ can be inferred from reduced sensitivity to painful stimuli. As described in the Examples below, animal models can be used to study the development, maintenance, and relief of chronic neuropathic or inflammatory pain. Animals subjected to these models generally develop thermal hyperalgesia (i.e., an increased response to a stimulus that is normally painful) and/or allodynia (i.e., pain due to a stimulus that is not normally painful). Sensitivity to mechanical and thermal stimuli can be assessed (see Bennett, Methods in Pain Research, pp. 67-91, Kruger, Ed. (2001)) to evaluate the effectiveness of P2X₃ antisense treatment.

Methods for formulating and subsequently administering therapeutic compositions are well known to those skilled in the art. See, for example, Remington, The Science and Practice of Pharmacy, 20^{th} Ed., Gennaro & Gennaro, eds., Lippincott, Williams & Wilkins (2000). Dosing is generally dependent on the severity and responsiveness of the disease state to be treated, with the course of treatment lasting from several days to several months, or until a cure is effected or a diminution of the disease state is achieved. Persons of ordinary skill in the art routinely determine optimum dosages, dosing methodologies and repetition rates. Optimum dosages can vary depending on the relative potency of individual oligonucleotides, and can generally be estimated based on EC₅₀ found to be effective in in vitro and in vivo animal models. Typically, dosage is from 0.01 μg to 100 g per kg of body weight, and may be given once or more daily, weekly, or even less often. Following successful treatment, it may be desirable to have the patient undergo maintenance therapy to prevent the recurrence of the disease state.

The present invention provides pharmaceutical compositions and formulations that include the P2X₃ antisense oligonucleotides of the invention. P2X₃ antisense oligonucleotides therefore can be admixed, encapsulated, conjugated or otherwise associated with other molecules, molecular structures, or mixtures of oligonucleotides such as, for example, liposomes, receptor targeted molecules, or oral, rectal, topical or other formulations, for assisting in uptake, distribution and/or absorption.

A “pharmaceutically acceptable carrier” (also referred to herein as an “excipient”) is a pharmaceutically acceptable solvent, suspending agent, or any other pharmacologically inert vehicle for delivering one or more therapeutic compounds (e.g., P2X₃ antisense oligonucleotides) to a subject. Pharmaceutically acceptable carriers can be liquid or solid, and can be selected with the planned manner of administration in mind so as to provide for the desired bulk, consistency, and other pertinent transport and chemical properties, when combined with one or more of therapeutic compounds and any other components of a given pharmaceutical composition. Typical pharmaceutically acceptable carriers that do not deleteriously react with nucleic acids include, by way of example and not limitation: water; saline solution; binding agents (e.g., polyvinylpyrrolidone or hydroxypropyl methylcellulose); fillers (e.g., lactose and other sugars, gelatin, or calcium sulfate); lubricants (e.g., starch, polyethylene glycol, or sodium acetate); disintegrates (e.g., starch or sodium starch glycolate); and wetting agents (e.g., sodium lauryl sulfate).

The pharmaceutical compositions of the present invention can be administered by a number of methods depending upon whether local or systemic treatment is desired and upon the area to be treated. Administration can be, for example, topical (e.g., transdermal, ophthalmic, or intranasal); pulmonary (e.g., by inhalation or insufflation of powders or aerosols); oral; or parenteral (e.g., by subcutaneous, intrathecal, intraventricular, intramuscular, or intraperitoneal injection, or by intravenous drip). Administration can be rapid (e.g., by injection) or can occur over a period of time (e.g., by slow infusion or administration of slow release formulations). For treating tissues in the central nervous system, antisense oligonucleotides can be administered by injection or infusion into the cerebrospinal fluid, preferably with one or more agents capable of promoting penetration of the antisense oligonucleotide across the blood-brain barrier.

Formulations for topical administration of antisense oligonucleotides include, for example, sterile and non-sterile aqueous solutions, non-aqueous solutions in common solvents such as alcohols, or solutions in liquid or solid oil bases. Such solutions also can contain buffers, diluents and other suitable additives. Pharmaceutical compositions and formulations for topical administration can include transdermal patches, ointments, lotions, creams, gels, drops, suppositories, sprays, liquids, and powders. Coated condoms, gloves and the like also may be useful. Conventional pharmaceutical carriers, aqueous, powder or oily bases, thickeners and the like may be necessary or desirable.

Compositions and formulations for oral administration include, for example, powders or granules, suspensions or solutions in water or non-aqueous media, capsules, sachets, or tablets. Such compositions also can incorporate thickeners, flavoring agents, diluents, emulsifiers, dispersing aids, or binders. Oligonucleotides with at least one 2′-O-methoxyethyl modification (described above) may be particularly useful for oral administration.

Compositions and formulations for parenteral, intrathecal or intraventricular administration can include sterile aqueous solutions, which also can contain buffers, diluents and other suitable additives (e.g., penetration enhancers, carrier compounds and other pharmaceutically acceptable carriers).

Pharmaceutical compositions of the present invention include, but are not limited to, solutions, emulsions, aqueous suspensions, and liposome-containing formulations. These compositions can be generated from a variety of components that include, for example, preformed liquids, self-emulsifying solids and self-emulsifying semisolids. Emulsions are often biphasic systems comprising of two immiscible liquid phases intimately mixed and dispersed with each other; in general, emulsions are either of the water-in-oil (w/o) or oil-in-water (o/w) variety. Emulsion formulations have been widely used for oral delivery of therapeutics due to their ease of formulation and efficacy of solubilization, absorption, and bioavailability.

Liposomes are vesicles that have a membrane formed from a lipophilic material and an aqueous interior that can contain the antisense composition to be delivered. Liposomes can be particularly useful from the standpoint of drug delivery due to their specificity and the duration of action they offer. Liposome compositions can be formed, for example, from phosphatidylcholine, dimyristoyl phosphatidylcholine, dipalmitoyl phosphatidylcholine, dimyristoyl phosphatidylglycerol, or dioleoyl phosphatidylethanolamine. Numerous lipophilic agents are commercially available, including Lipofectin® (Invitrogen/Life Technologies, Carlsbad, Calif.) and Effectene™ (Qiagen, Valencia, Calif.).

The P2X₃ antisense oligonucleotides of the invention further encompass any pharmaceutically acceptable salts, esters, or salts of such esters, or any other compound which, upon administration to an animal including a human, is capable of providing (directly or indirectly) the biologically active metabolite or residue thereof Accordingly, for example, the invention provides pharmaceutically acceptable salts of P2X₃ antisense oligonucleotides, prodrugs and pharmaceutically acceptable salts of such prodrugs, and other bioequivalents. The term “prodrug” indicates a therapeutic agent that is prepared in an inactive form and is converted to an active form (i.e., drug) within the body or cells thereof by the action of endogenous enzymes or other chemicals and/or conditions. The term “pharmaceutically acceptable salts” refers to physiologically and pharmaceutically acceptable salts of the oligonucleotides of the invention (i.e., salts that retain the desired biological activity of the parent oligonucleotide without imparting undesired toxicological effects). Examples of pharmaceutically acceptable salts of oligonucleotides include, but are not limited to, salts formed with cations (e.g., sodium, potassium, calcium, or polyamines such as spermine); acid addition salts formed with inorganic acids (e.g., hydrochloric acid, hydrobromic acid, sulfuric acid, phosphoric acid, or nitric acid); salts formed with organic acids (e.g., acetic acid, citric acid, oxalic acid, palmitic acid, or fumaric acid); and salts formed from elemental anions (e.g., chlorine, bromine, and iodine).

Pharmaceutical compositions containing the antisense oligonucleotides of the present invention also can incorporate penetration enhancers that promote the efficient delivery of nucleic acids, particularly oligonucleotides, to the skin of animals. Penetration enhancers can enhance the diffusion of both lipophilic and non-lipophilic drugs across cell membranes. Penetration enhancers can be classified as belonging to one of five broad categories, i.e., surfactants (e.g., sodium lauryl sulfate, polyoxyethylene-9-lauryl ether and polyoxyethylene-20-cetyl ether); fatty acids (e.g., oleic acid, lauric acid, myristic acid, palmitic acid, and stearic acid); bile salts (e.g., cholic acid, dehydrocholic acid, and deoxycholic acid); chelating agents (e.g., disodium ethylenediaminetetraacetate, citric acid, and salicylates); and non-chelating non-surfactants (e.g., unsaturated cyclic ureas).

Certain embodiments of the invention provide pharmaceutical compositions containing (a) one or more antisense oligonucleotides and (b) one or more other agents that function by a non-antisense mechanism. For example, anti-inflammatory drugs, including but not limited to nonsteroidal anti-inflammatory drugs and corticosteroids, and antiviral drugs, including but not limited to ribivirin, vidarabine, acyclovir and ganciclovir, can be included in compositions of the invention. Other non-antisense agents (e.g., chemotherapeutic agents) are also within the scope of this invention. Such combined compounds can be used together or sequentially.

The antisense compositions of the present invention additionally can contain other adjunct components conventionally found in pharmaceutical compositions. Thus, the compositions also can include compatible, pharmaceutically active materials such as, for example, antipruritics, astringents, local anesthetics or anti-inflammatory agents, or additional materials useful in physically formulating various dosage forms of the compositions of the present invention, such as dyes, flavoring agents, preservatives, antioxidants, opacifiers, thickening agents and stabilizers. Furthermore, the composition can be mixed with auxiliary agents, e.g., lubricants, preservatives, stabilizers, wetting agents, emulsifiers, salts for influencing osmotic pressure, buffers, colorings, flavorings, and aromatic substances. When added, however, such materials should not unduly interfere with the biological activities of the antisense components within the compositions of the present invention. The formulations can be sterilized and, if desired, and the like which do not deleteriously interact with the nucleic acid(s) of the formulation.

The pharmaceutical formulations of the present invention, which can be presented conveniently in unit dosage form, can be prepared according to conventional techniques well known in the pharmaceutical industry. Such techniques include the step of bringing into association the active ingredients (e.g., the P2X₃ antisense oligonucleotides of the invention) with the desired pharmaceutical carrier(s) or excipient(s). Typically, the formulations can be prepared by uniformly bringing the active ingredients into intimate association with liquid carriers or finely divided solid carriers or both, and then, if necessary, shaping the product. Formulations can be sterilized if desired, provided that the method of sterilization does not interfere with the effectiveness of the antisense oligonucleotide contained in the formulation.

The compositions of the present invention can be formulated into any of many possible dosage forms such as, but not limited to, tablets, capsules, liquid syrups, soft gels, suppositories, and enemas. The compositions of the present invention also can be formulated as suspensions in aqueous, non-aqueous or mixed media. Aqueous suspensions further can contain substances that increase the viscosity of the suspension including, for example, sodium carboxymethylcellulose, sorbitol, and/or dextran. Suspensions also can contain stabilizers.

Nucleic Acid Constructs

Nucleic acid constructs (e.g., a plasmid vector) are capable of transporting a nucleic acid into a host cell. Suitable host cells include prokaryotic or eukaryotic cells (e.g., bacterial cells such as E. coli, insect cells, yeast cells, and mammalian cells). Some constructs are capable of autonomously replicating in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell and are replicated with the host genome.

Nucleic acid constructs can be, for example, plasmid vectors or viral vectors (e.g., replication defective retroviruses, adenoviruses, and adeno-associated viruses). Nucleic acid constructs include one or more regulatory sequences operably linked to the nucleic acid of interest (e.g., a nucleic acid encoding a transcript that specifically hybridizes to a P2X₃ mRNA in its native form). With respect to regulatory elements, “operably linked” means that the regulatory sequence and the nucleic acid of interest are positioned such that nucleotide sequence is transcribed (e.g., when the vector is introduced into the host cell).

Regulatory sequences include promoters, enhancers and other expression control elements (e.g., polyadenylation signals). (See, e.g., Goeddel, Gene Expression Technology: Methods in Enzymology, 185, Academic Press, San Diego, Calif. (1990)). Regulatory sequences include those that direct constitutive expression of a nucleotide sequence in many types of host cells and that direct expression of the nucleotide sequence only in certain host cells (e.g., cell type or tissue-specific regulatory sequences).

Articles of Manufacture

Antisense oligonucleotides of the invention can be combined with packaging material and sold as kits of reducing P2X₃ expression. Components and methods for producing articles of manufacture are well known. The articles of manufacture may combine one or more of the antisense oligonucleotides set out in the above sections. In addition, the article of manufacture further may include buffers, hybridization reagents, or other control reagents for reducing or monitoring reduced P2X₃ expression. Instructions describing how the antisense oligonucleotides are effective for reducing P2X₃ expression can be included in such kits.

The invention will be further described in the following examples, which do not limit the scope of the invention described in the claims.

### EXAMPLES

### Example 1

### Materials and Methods

Determination of Accessible Sites within the P2X₃ mRNA and Design of P2X₃ Antisense Oligonucleotides

Antisense oligonucleotides were designed to be targeted to accessible regions of the rat or human P2X₃ mRNA (as determined by the RiboTAG™ method). Accessible regions of rat and human P2X₃ are shown in Table 1. Antisense oligonucleotides were commercially synthesized (Midland Certified Reagent Company, Midland, Tex.) and purified prior to injection.

TABLE 1
Accessible sequences within P2X₃ mRNA

| | |
|---|---|
| Rat P2X₃ | Human P2X₃ |

| Start | End | Start | End |
|---|---|---|---|
| 68 | 88 | 7 | 29 |
| 209 | 230 | 95 | 105 |
| 235 | 247 | 207 | 217 |
| 285 | 296 | 221 | 240 |
| 346 | 355 | 248 | 258 |
| 383 | 406 | 278 | 293 |
| 490 | 512 | 338 | 365 |
| 530 | 543 | 471 | 482 |
| 553 | 565 | 486 | 502 |
| 649 | 658 | 544 | 562 |
| 665 | 679 | 747 | 761 |
| 727 | 739 | 784 | 796 |
| 756 | 779 | 815 | 850 |
| 817 | 856 | 865 | 879 |
| 874 | 912 | 883 | 905 |
| 959 | 991 | 922 | 932 |
| 1028 | 1050 | 953 | 968 |
| 1087 | 1116 | 985 | 1000 |
| 1145 | 1177 | 1033 | 1044 |
| 1237 | 1256 | 1156 | 1170 |
| 1266 | 1281 | 1239 | 1261 |
| 1297 | 1307 | 1297 | 1314 |
| 1314 | 1334 | 1411 | 1439 |
| 1339 | 1359 | | |
| 1434 | 1463 | | |
| 1523 | 1535 | | |
| 1630 | 1646 | | |
| 1677 | 1688 | | |
| 1729 | 1741 | | |

Methods for Evaluating Pain in Rats Treated with Antisense P2X₃

Two different models of chronic pain were used to evaluate the effects of P2X₃ knock-down by intrathecally administered antisense oligonucleotides. Both models included the following six steps (described in greater detail below):

(1) spinal catheterization;

(2) nociceptive testing (baseline);

(3) induction of chronic neuropathic or inflammatory pain;

(4) nociceptive testing (post-injury);

(5) antisense injection; and

(6) nociceptive testing (post-treatment).

Spinal Catheterization: Male Sprague Dawley rats weighing between 120 and 150 g were obtained from Harlan (Indianapolis, Ind.). Rats were deeply anesthetized with a mixture containing 75 mg/kg ketamine, 5 mg/kg xylazine, and 1 mg/kg acepromazine, and a catheter (8.5 cm; PE-10) was passed to the lumbosacral intrathecal space through an incision in the dura over the atlantooccipital joint. Animals were allowed to recover for 3 days before being subjected to models of chronic pain.

Mechanical Nociceptive Testing: Baseline, post-injury, and post-treatment values for mechanical sensitivity were evaluated with calibrated monofilaments (von Frey filaments) according to the up-down method (Chaplan, et al. (1994) J. Neurosci. Methods 53:55-63). Animals were placed on a wire mesh platform and allowed to acclimate to their surroundings for a minimum of 10 minutes before testing. Filaments of increasing force were sequentially applied to the plantar surface of the paw just to the point of bending, and held for three seconds. The behavioral endpoint of the stimulus (achieved when the stimulus was of sufficient force) was the point at which the animal would lick, withdraw and/or shake the paw. The force or pressure required to cause a paw withdrawal was recorded as a measure of threshold to noxious mechanical stimuli for each hind-paw. The mean and standard error of the mean (SEM) were determined for each hind-paw in each animal in each treatment group. Repeated measures ANOVA followed by the Bonferonni post-hoc test were used to determine significance. Since this stimulus is normally not considered painful and rats do not normally respond to filaments in the range selected, significant injury-induced increases in responsiveness in this test were interpreted as a measure of mechanical allodynia.

Thermal Nociceptive Testing: Baseline, post-injury, and post-treatment thermal sensitivities were determined by measuring withdrawal latencies in response to radiant heat stimuli delivered to the plantar surface of the hind-paws (Hargreaves et al. (1988), Pain 32:77-88). Animals were placed on a plexiglass platform and allowed to acclimate for a minimum of 10 minutes. A radiant heat source was directed to the plantar surface, and the time to withdrawal was measured. For each paw, the withdrawal latency was determined by averaging three measurements separated by at least 5 minutes. The heating device was set to automatically shut off after a programmed period of time to avoid damage to the skin of unresponsive animals. The data were analyzed using repeated measures ANOVA followed by the Bonferonni post-hoc test. Significant injury-induced increases in thermal response latencies were considered to be a measure of thermal hyperalgesia since the stimulus is normally in the noxious range.

Induction of Chronic Neuropathic Pain: The Spinal Nerve Ligation (SNL) model (Kim and Chung (1992) Pain 50:355-363) was used to induce chronic neuropathic pain. Rats were anesthetized with isoflurane, the L5 transverse process was removed, and the L5 and L6 spinal nerves were tightly ligated with 6-0 silk suture. The wound was then closed with internal sutures and external staples. Following surgery, animals were kept on a warming blanket and were periodically turned and carefully observed until complete recovery from anesthesia was obtained. Sham surgery consisted of removing the transverse process and exposing the L5 spinal nerve without ligating. All operations were performed on the left side.

Induction of Chronic Inflammation: The complete freunds adjuvant (CFA) model of chronic peripheral inflammation was utilized (see, for example, Hylden et al. (1989) Pain 37:229-243). Rats under isoflurane anesthesia received an injection of CFA (75 μl) into the left hindpaw using a sterile 1.0 ml syringe. A separate population of control rats was subjected to unilateral injection of saline.

Antisense Injection: Oligonucleotides were dissolved in dH₂O and delivered into the intrathecal space in a volume of 5 μl per injection as previously described (see, for example, Bilsky et al. (1996) Neurosci. Lett., 220:155-158; Bilsky et al. (1996) J. Pharmacol. Exp. Ther., 277:491-501; and Vanderah et al. (1994) Neuroreport., 5:2601-2605). Antisense oligonucleotides were administered twice daily for 3 to 4 days, beginning on the afternoon following post-injury (baseline) nociceptive testing. Antisense oligonucleotides included the sequences 5′-GAG GTT TCC CTT CTC AAA-3′, 5′-ATG TCC TTG TCG GTG AGG TTA GG-3′, and 5′-CTA GTC TTT GGG GTG AAC-3′, which specifically hybridize to nucleotides 721 through 744, 747 through 770, and 1478 through 1495, respectively, of SEQ ID NO:1. Random oligonucleotides were used as controls. Random oligonucleotides were used as controls.

Immunolocalization of P2X₃

Spinal cord tissue was obtained post-mortem and immersion-fixed overnight in 4% paraformaldehyde. After fixation, the tissue was washed in phosphate buffered saline (PBS) for 2 to 3 days and stored in 10% sucrose solution. The spinal cord was sliced into 14 μm sections using a cryostat. Slide-mounted tissue sections were incubated in blocking buffer for 1 hour at room temperature, followed by incubation with primary antisera (guinea pig anti-P2X₃, 1:5000) overnight at 4° C. Staining was visualized using biotinylated tiramine amplification as previously described (Vulchanova et al. (1997) Neuropharmacology 36:1229-1242). For absorption control, the primary antisera were incubated with the corresponding peptide antigen (10 μg/ml) prior to application to tissue sections.

Dorsal root ganglia were obtained post-mortem and frozen in liquid nitrogen. The frozen tissue was cut into 10 μm sections, which were thaw-mounted on cooled gelatin-coated slides. Sections were fixed with paraformaldehyde-picric acid fixative for 30 minutes immediately before processing for immunohistochemistry. The slide-mounted tissue sections were incubated in blocking buffer for 1 hour at room temperature, followed by incubation with primary antisera (guinea pig anti-P2X₃, 1:500) overnight at 4° C. Slides were washed three times in PBS, incubated with secondary antisera for 1 hour at room temperature, washed again and coverslipped. For absorption control, the primary antisera were incubated with the corresponding peptide antigen (10 μg/ml) prior to applying to tissue sections. Staining was visualized with cyanine 3.18-conjugated secondary antisera (Jackson ImmunoResearch, West Grove, Calif.).

### Example 2

### Antisense Knockdown of P2X3 in Rat Spinal Cord Supports a Role in Chronic Neuropathic and Inflammatory Pain

Antisense oligonucleotides were designed by the RiboTAG™ method and used to evaluate the role of P2X₃ in chronic pain. Thermal (radiant heat) and mechanical (von Frey) pain thresholds were obtained before and after induction of chronic pain (neuropathic or inflammatory, as described in Example 1, above). Antisense oligonucleotides or randomized controls were delivered twice daily for 3 to 4 days, and thermal and mechanical thresholds were reassessed.

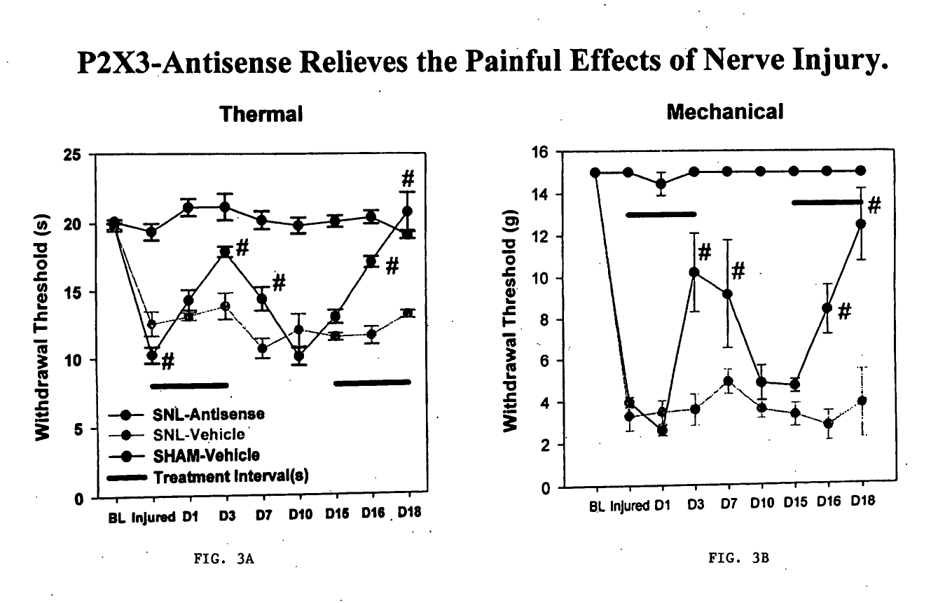

FIG. 3A shows that animals were significantly more sensitive to thermal stimuli following nerve injury by spinal nerve ligation (as evidenced by the decreases in their response thresholds compared to pre-injury baseline (BL) and uninjured controls). Treatment with P2X₃ antisense oligonucleotides between days 0 and 3 significantly relieved the sensitivity to thermal stimulation, as compared to treatment with a control vehicle. Cessation of treatment between days 3 and 15 caused a return to maximal sensitivity, which was again relieved by the resumption of antisense treatment between days 15 and 18. Repeat injections of vehicle only had no effect.

Similarly, FIG. 3B shows that nerve-injured animals also were significantly more sensitive to mechanical stimuli, and that P2X₃ antisense treatment reversibly relieved this sensitivity.

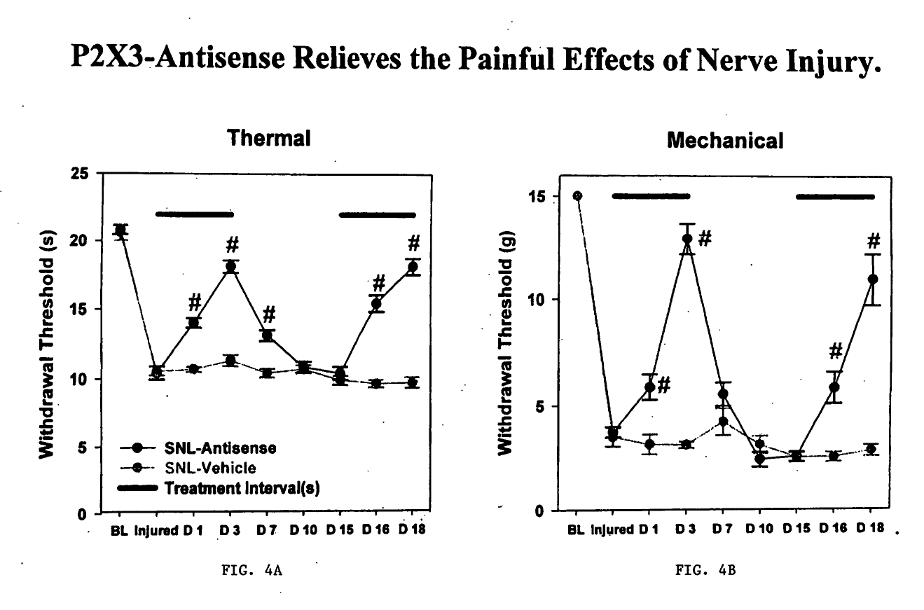

FIG. 4A and FIG. 4B depict the results of analagous experiments using a separate group of animals. As in the previous experiments, treatment of chronic pain with P2X₃ antisense oligonucleotides reversibly alleviated the sensitivity to thermal and mechanical stimuli.

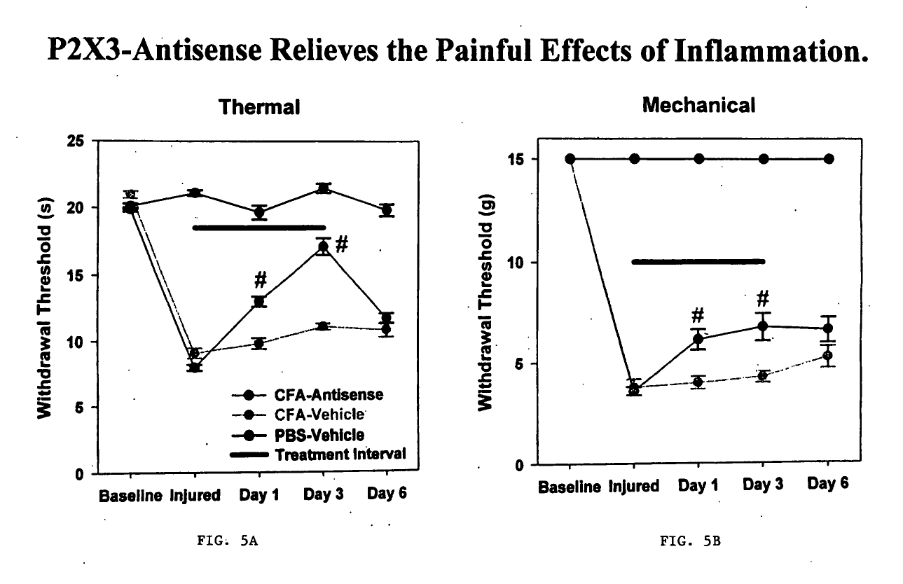

As shown in FIG. 5A and FIG. 5B, animals subjected to inflammation also were significantly more sensitive to thermal and mechanical stimuli (as evidenced by the decreases in their response thresholds compared to pre-inflammation baseline (BL) and uninflamed controls).

Animals from several experiments were euthanized so that levels of P2X₃ could be assessed by immunostaining. Rats treated with the P2X₃ antisense oligonucleotides displayed lower levels of P2X₃ in dorsal horn spinal cord tissue than control animals treated with randomized oligonucleotides.

### Example 3

### Immunolocalization of human P2X₃

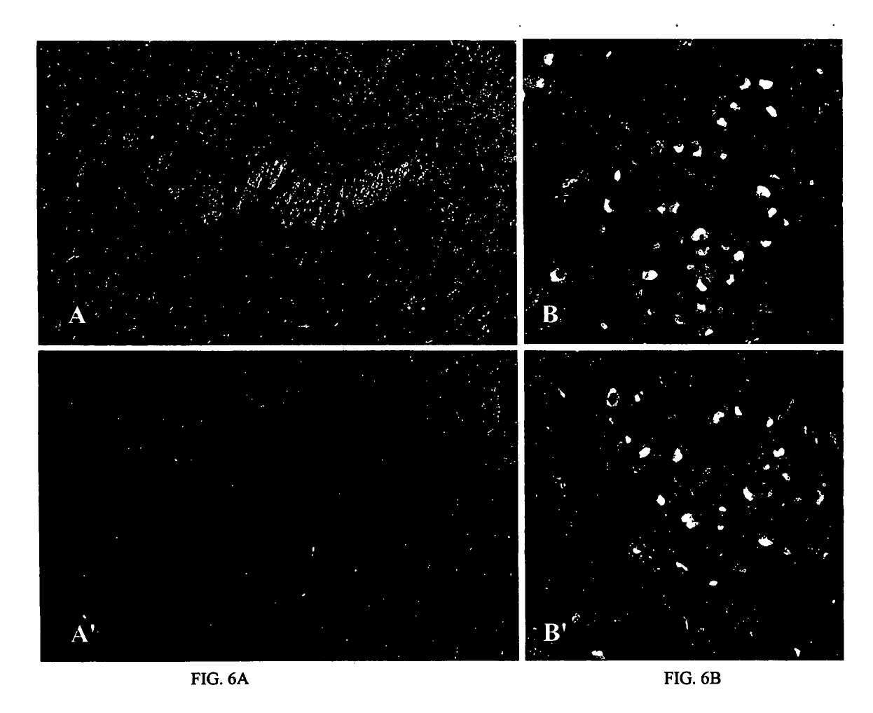

P2X₃ immunoreactivity in human spinal cord was restricted to a band corresponding to inner lamina II (FIG. 6A and 6A′). In dorsal root ganglia, P2X₃ immunoreactivity was present in a subset of small neurons (FIG. 6B and 6B′). Specificity of the labeling in spinal cord and dorsal root ganglia was demonstrated by absorption controls with the peptide antigen (A′ and B′, respectively). This localization of human P2X₃ was identical to the localization of rat P2X₃, which supports the pursuit of P2X₃ as a therapeutic target for chronic pain.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An isolated antisense oligonucleotide consisting essentially of 10 to 50 nucleotides, wherein said oligonucleotide specifically hybridizes within an accessible region, said region defined by nucleotides 68 through 88, 209 through 230, 235 through 247, 285 through 296, 346 through 355, 383 through 406, 490 through 512, 530 through 543, 553 through 565, 649 through 658, 665 through 679, 727 through 739, 756 through 779, 817 through 856, 874 through 912, 959 through 991, 1028 through 1050, 1087 through 1116, 1145 through 1177, 1237 through 1256, 1266 through 1281, 1297 through 1307, 1314 through 1334, 1339 through 1359, 1434 through 1463, 1523 through 1535, 1630 through 1646, 1677 through 1688, or 1729 through 1741 of SEQ ID NO:1, and wherein said oligonucleotide inhibits the production of P2X₃.

2. The isolated antisense oligonucleotide of claim 1, wherein said oligonucleotide specifically hybridizes within an accessible region defined by nucleotides 383 through 406, 756 through 779, 490 through 512, or 727 through 739 of SEQ ID NO:1.

3. The isolated antisense oligonucleotide of claim 2, wherein said oligonucleotide specifically hybridizes within an accessible region defined by nucleotides 384 through 397, 766 through 775, 495 through 510, or 732 through 736 of SEQ ID NO:1.

4. The isolated antisense oligonucleotide of claim 1, wherein said oligonucleotide specifically hybridizes within an accessible region defined by nucleotides 1434 through 1463, 1237 through 1256, 959 through 991, or 1028 through 1050 of SEQ ID NO:1.

5. The isolated antisense oligonucleotide of claim 1, wherein said oligonucleotide specifically hybridizes within an accessible region defined by nucleotides 817 through 856, 553 through 565, 285 through 296, 209 through 230, or 1145 through 1177 of SEQ ID NO:1.

6. The isolated antisense oligonucleotide of claim 1, wherein said oligonucleotide specifically hybridizes within an accessible region defined by nucleotides 383 through 404, 721 through 744, 747 through 770, or 1314 through 1344 of SEQ ID NO:1.

7. A composition comprising the isolated antisense oligonucleotide of claim 1.

8. The composition of claim 7, wherein said composition comprises a plurality of isolated antisense oligonucleotides, wherein each antisense oligonucleotide specifically hybridizes within a different accessible region.

9. An isolated antisense oligonucleotide consisting essentially of 10 to 50 nucleotides, wherein said oligonucleotide specifically hybridizes within an accessible region, said region defined by nucleotides 7 through 29, 95 through 105, 207 through 217, 221 through 240, 248 through 258, 278 through 293, 338 through 365, 471 through 482, 486 through 502, 544 through 562, 747 through 761, 784 through 796, 815 through 850, 865 through 879, 883 through 905, 922 through 932, 953 through 968, 985 through 1000, 1033 through 1044, 1156 through 1170, 1239 through 1261, 1297 through 1314, or 1411 through 1439 of SEQ ID NO:2, and wherein said isolated antisense oligonucleotide inhibits the production of P2X₃.

10. The isolated antisense oligonucleotide of claim 9, wherein said antisense oligonucleotide specifically hybridizes within an accessible region defined by nucleotides 953 through 968, 1297 through 1314, or 815 through 850 of SEQ ID NO:2.

11. The isolated antisense oligonucleotide of claim 10, wherein said antisense oligonucleotide specifically hybridizes within an accessible region defined by nucleotides 957 through 967, 1297 through 1301, or 817 through 823 of SEQ ID NO:2.

12. The isolated antisense oligonucleotide of claim 9, wherein said antisense oligonucleotide specifically hybridizes within an accessible region defined by nucleotides 747 through 761, 985 through 1000, or 486 through 502 of SEQ ID NO:2.

13. The isolated antisense oligonucleotide of claim 9, wherein said antisense oligonucleotide specifically hybridizes within an accessible region defined by nucleotides 338 through 365, 278 through 293, 544 through 562, or 221 through 240 of SEQ ID NO:2.

14. The isolated antisense oligonucleotide of claim 9, wherein said antisense oligonucleotide specifically hybridizes within an accessible region defined by nucleotides 484 through 501 or 742 through 762 of SEQ ID NO:2.

15. A composition comprising the isolated antisense oligonucleotide of claim 9.

16. The composition of claim 15, wherein said composition comprises a plurality of isolated antisense oligonucleotides, wherein each antisense oligonucleotide specifically hybridizes with a different accessible region.

17. An isolated oligonucleotide consisting essentially of the sequence of SEQ ID NO:3, SEQ ID NO:4, SEQ ID NO:5, SEQ ID NO:6, SEQ ID NO:7, or SEQ ID NO:8.

18. A method of decreasing production of P2X₃ in cells or tissues, comprising contacting said cells or tissues with an antisense oligonucleotide that specifically hybridizes within an accessible region of P2X₃ provided that said accessible region is not a region defined by nucleotides 1279 through 1296 or 1315 through 1334 of SEQ ID NO:2.

19. The method of claim 18, wherein said contacting step results in an inhibition of pain sensory neurons.

20. The method of claim 18, wherein said contacting step results in increased bladder capacity.

21. A nucleic acid construct comprising a regulatory element operably linked to a nucleic acid encoding a transcript, wherein said transcript specifically hybridizes within one or more accessible regions of P2X₃ mRNA in its native form.

22. A host cell comprising the nucleic acid construct of claim 21.

23. An isolated antisense oligonucleotide that specifically hybridizes within an accessible region of P2X₃ mRNA in its native form, provided that said accessible region is not a region defined by nucleotides 1279 through 1296 or 1315 through 1334 of SEQ ID NO:2, and wherein said antisense oligonucleotide inhibits production of P2X₃.
